(19) United States
(12) Patent Application Publication
(10) Pub. No.: US 2003/0032996 A1
(43) Pub. Date: Feb. 13, 2003

(54) Cryogenic massage tube and compress

(76) Inventor: Arlan Jay Hallman, Plantation, FL (US)

Correspondence Address: Melvin K. Silverman, Suite 440, 4901 North Federal Highway, Fort Lauderdale, FL, 33308, US

(21) Appl. No.: 10/214,892

(22) Filed: Aug. 7, 2002

Related U.S. Application Data
(60) Provisional application No. 60/311,052, filed on Aug. 8, 2001.

(51) Int. Cl. A61F 7/00

(52) U.S. Cl. 607/108; 607/109; 607/114

(57) ABSTRACT

A cryogenic therapeutic massage Tube includes a pressure-deformable gel, which may be cooled by freezing, and which is permanently encased in a flexible thermally conductive material to substantially define a cylindrical compress having a diameter of about 4 to 7 inches and a ratio of length-to-diameter in a range of about 2 to about 4. The deformable cylindrical compress is proportioned to engage a plurality of anatomic curves including curves of the neck, back, feet and hands, and to impart thereto a soothing, cooling and pain relieving effect, in which such tube or cylinder of cryogenic gel may be provided within a thick cloth sack to preclude skin burn and absorb moisture from the surface of the skin against which the compress is applied.

### REFERENCE TO RELATED APPLICATION

This application is a non-provisional, utility conversion of Provisional Application No. 60/311,052, filed Aug. 8, 2001, and the same is hereby incorporated in full by reference.

Not applicable.

### BACKGROUND OF THE INVENTION

1. Area of Invention

This invention relates to therapeutic pillows and rolls formed of a cryogenic material.

2. Prior Art

Hot and cold therapeutic pillows for the support of the head and neck are known as, for example, is reflected in U.S. Pat. No. 5,545,199 (1996) to Hudson, entitled Hot and Cold Therapeutic Pillow. Such devices are typically formed of a gel pack which may be reduced in temperature by freezing and which, in turn, is embedded in a deformable material to impart to the structure the contour of a support of the neck and head. Cryogenic spheres are known for application of the skin to produce vasoconstrictive and vasodilative effects which are beneficial to maintenance of epidermal tissues of the skin being massaged or treated. See for example U.S. Pat. No. 5,127,395 (1992) to Bontemps, entitled Cryogenic Device for Skin Massage. The prior art is also reflected in the use of small cryogenic massage tools as, for example, is shown in U.S. Pat. No. 4,745,909 (1988) to Pelton, et al, entitled Cold Massage Tool and Method of Use Thereof.

So-called ice massage has been known for some time as is set forth in a study by Melzak et al in 1980 in which patients suffering from acute dental pain were treated with ice massage of the back of the hand, on the same side of the body as the dental pain, employing what is known as the Hoku acupuncture point. Ice massage has been found to substantially reduce the intensity of dental pain for most patients and to be more effective than tactile massage toward this end. These observations led to a further study by Melzak et al correlating the effectiveness of ice massage with the use of TENS, a form of electrotherapy. In this study, ice massage was found to be at least as effective as TENS in the treatment of many pain conditions. Accordingly, it has been hypothesized that ice can be used in lieu of acupuncture if applied with sufficient pressure at the correct acupuncture or trigger point.

The application of ice massage in which ice cubes are held within gauze pads or within strips of cloth, and then moved in a circular motion upon a painful area, has been known and used in the West for decades.

A clinical study by Grant (1964), based upon more than 7,000 out patients, describes the value of direct application of ice for shoulder-neck and lower back pain. In a discovery based upon clinical observations in the 1930s, Dr. Janet Travell (later personal physician to President Kennedy) discovered that musceloskeletal pains associated with localized points (said “trigger points”) were highly sensitive to touch. However, said Travell found that by dry massaging of a given spot or the application of cold spray thereto, it was possible to relieve the pain of the trigger point as well as pain associated with the larger area of referred pain.

Thus, it has been found that ice massage is a useful procedure which can be used for a variety of clinical pain problems. Therein, the mechanisms responsible for the effects of ice massage appear to flow from several effects:

1. Production of local constriction of blood vessels and consequently may diminish swelling and slow the release of histamines and other pain-eliciting substances.

2. Reduction of interaction among nerve fibers of different sizes such as exists at the dorsal perimeter of the spinal column, by reducing the intensity.

3. Areas of the brain stem can, when cooled, exert an inhibitory control over neck pain and headaches of various types.

While so-called freezable gels, formed of such materials as sodium polyacrylate, which may be readily reduced in temperature by placement within a freezer, have become well known, a practically usable therapeutic compress, ergonomically adapted to a plurality of physiologic interfaces, has not appeared in the art. For example, said references to Pelton and Bontemps are devices which contemplate use by a masseur or clinician upon a patient, whereas references such as Hudson above are intended for static use to cradle the neck and head when the user is reading, resting, viewing television, or the like.

In distinction, the present inventive cryogenic tube is one adapted for dynamic self-use upon many parts of the human body, as is more fully set forth below.

### SUMMARY OF THE INVENTION

The present cryogenic therapeutic system includes a pressure-deformable gel which may be cooled by freezing and which is permanently encased in a flexible thermally conductive material to define a substantially cylindrical compress having a diameter of about 4 to 7 inches and a ratio of length-to-diameter in a range of about 2 to about 4. A deformable radius of said cylindrical compress is thereby proportioned to engage a plurality of anatomic curves including curves of the neck, back, feet and hands, and to impart thereto a soothing, cooling and pain-relieving effect and to address miscurvatures of the neck and back. Such tube or cylinder of the cryogenic gel may be provided within a thick cloth sack to thereby preclude skin burn and absorb moisture from the surface of the skin against which the compress is applied.

The principal object of the present invention is therefore to expand and improve the function of therapeutic pillows and cervical rolls.

It is another object to retain and enhance the comfort derivable from the therapeutic freeze-gel packs by providing the same in an ergonomic geometry useful in the treatment of many areas of the neck, back, feet and hands.

It is a further object to provide a cryogenic massage means to improve the effectiveness, durability and convenience of use of freeze gel packs and the like.

It is a still further object of the invention to provide a self-help means for the treatment of carpal tunnel syndrome, plantar/sole of the foot pain, cervical pain, lumbar pain, and relief of pain in large muscle groups.

It is a still further object to provide a cryogenic massage and treatment device for the provision of beneficial curvature and tract to areas which have been massaged.

The above and yet other objects and advantages of the present invention will become apparent from the hereinafter set forth Brief Description of the Drawings, Detailed Description of the Invention and claims appended herewith.

### BRIEF DESCRIPTION OF THE DRAWINGS

FIG. 1 is a perspective view of the cryogenic massage tube.

FIG. 2 is an operational view showing the use therewith to aid in treatment of pain of the cervical area and restoration of a proper cervical curve by tractioning of the neck.

FIG. 3 is an operational view of the inventive means showing use in a rolling action in the treatment of pain of the bottom or sole of the foot.

FIG. 4 is an operational view showing the use of the invention in connection with lumbar pain and in the restoration of proper curvature to the lumbar area of the spine.

FIG. 5 is an operational view of uses of the inventive means as an aid to restoration of proper curvature to the neck or lumbar area of the spine.

FIG. 6 is a further operational view showing use of the inventive therapeutic means in the treatment of arthritis, carpal tunnel syndrome, and related conditions.

### DETAILED DESCRIPTION OF THE INVENTION

With respect to the perspective view of FIG. 1, the inventive therapeutic system may be seen to include a tubular or cylindrical compress 10 having left and right bases 12, each having a diameter in a range of about 3 to about 7 inches in which a ratio of length-to-diameter of the structure so defined falls within a range within about 2 to about 4. A freezable gel which is permanently encased within a flexible, thermally conductive polymeric material, which forms the compress, may comprise any of a number of state-of-the-art ice gel materials including, for example, a powder of starch and sodium polyacrylate to which water is added to completely fill a bladder 13 defined by said flexible polymeric material. In a preferred embodiment, the bladder comprises two layers, including an inner layer having properties of low friction, non-adhering communication with said gel and a second layer adhered thereto having properties of biocompatibility with human skin. Therein, the inner layer may comprise a polyester while the outer layer may comprise a nylon or like material, both layers, in aggregate, defining a thickness of about 3 mils. It has been found that such deformable gel may be frozen within a freezer of a refrigerator and will then retain a temperature of below freezing for a period of more than five hours.

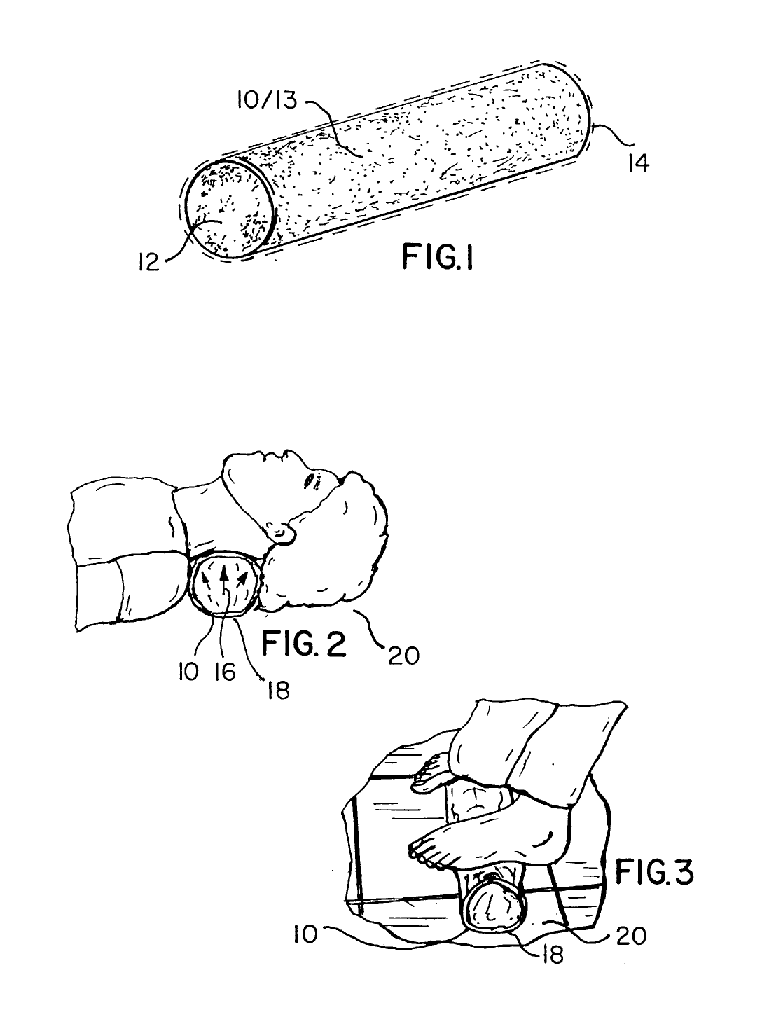

As is taught in references such as U.S. Pat. No. 5,545,199 above (which is incorporated herewith by reference), pressure-deformable freezable gel packs are well known in the art. However, the present invention lies in the discovery that if a tubular bladder is filled with such gel to a density in a range of about 0.17 to about 0.25 ounces per cubic inch, thereby producing a total weight in a range of 3.5 to 5.5 pounds, in which 4.5 pounds reflects a preferred embodiment, the resultant bladder will exhibit therapeutic properties at a radius, applied to the human body, in a range of 1.5 to 3.5 inches (corresponding to said diameter of said base 12 in a range of 3 to 7 inches). In other words, although freezable ice gel packs are well known in the art, ice packs having a density and geometry in the above set forth range are not known. By virtue of such density, which relates to the pressure at which the bladder is filled with the freezable gel and the inherent properties of the gel itself, the above set forth radii of base 12 have been found to be useful in treatment of a considerable range of muscular skeletal dysfunctions. These, in particular, are shown in FIGS. 2 through 6, and are as follows:

In FIG. 2 is shown use of the inventive system for treatment of cervical pain of any kind and, as well, to aid in the restoration of a proper curve of the cervical vertebrae of the spinal column. Also, appropriate pressure-deformable use of the present therapeutic means will, due to is particular radius and pressure deformability, produce a tractioning 16 of the neck when lying flat on floor 20 on one's back in the manner shown in FIGS. 2 and 5. Also, in FIG. 2 may be seen a Terry cloth sack 14 or the like which acts to preclude skin burn based from low temperature of the therapeutic means and to absorb moisture from the skin against which the compress 10 is applied.

In FIG. 3 is shown the use of the compress 10 in a rolling action for the treatment of pain of the sole of the foot, medically known as plantar fascaitis.

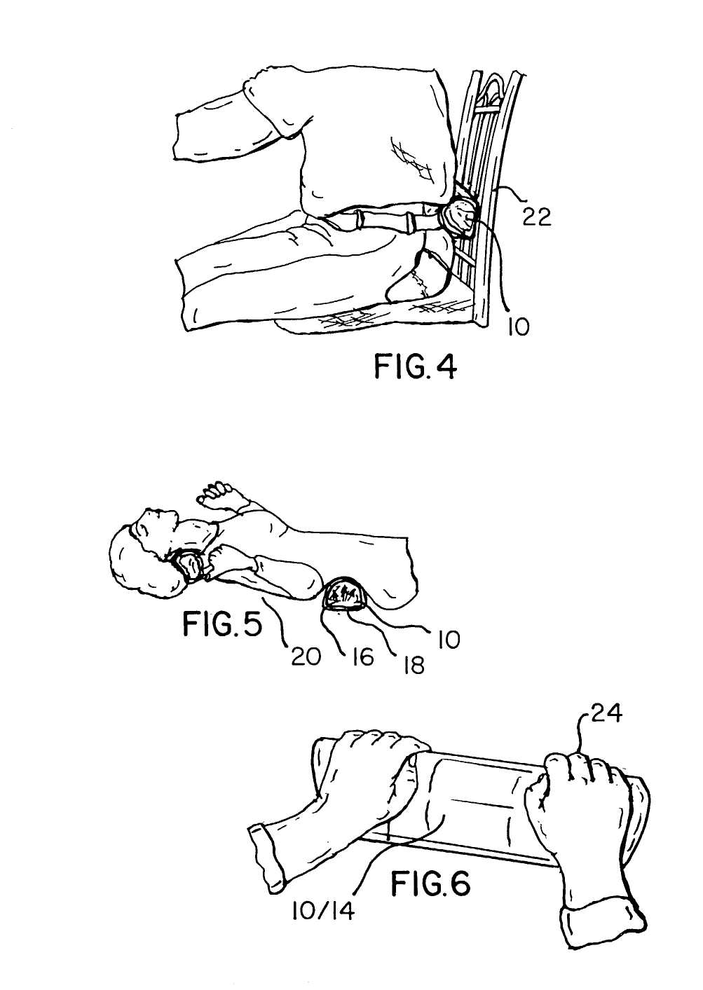

In FIG. 4 is shown the use of compress 10 positioned in the lumbar area of the back and against surface 22. Such use will relieve lumbar pain of any type and will assist in the restoration of a normal curvature to the lumbar vertebrate.

In FIG. 5 is shown another position in which compress 10 may be beneficially employed for the treatment of either neck or lumbar pain. Therein, the arrows represent the tractional force 16 which is applied to the lumbar curve because of the particularly ergonomic effect in the present therapeutic device given its above described ergonomic radius in a range of 1.5 to 3.5 inches. Therein bottom surface 18 of the compress 10 will, in the manner shown in FIGS. 2 to 5, conform to floor 20 or whatever surface is located opposite to the body area which is treated, such as a treatment surface of a therapy table. As such, the inventive compress conforms to both the anatomic and non-anatomic surfaces to which pressure is applied in order to produce desirable traction 16 in treatment of the above set forth conditions.

In FIG. 6 is shown use of the inventive compress 10 and its associated cloth carrier 14 to treat conditions of the hand including, without limitation, arthritis and carpal tunnel syndrome. Thereby fingers 24 and palms are able to engage and grip compress 10 in a rolling motion, similar to motion associated with treatment of the foot above shown and described with reference to FIG. 3. This treatment, it has been found, is most useful in relieving symptoms associated with various conditions of the hand and fingers.

It is, accordingly, to be appreciated that the present system, in addition to its utility in the reduction of inflammation and its analgesic effect in the application of cold to acupuncture points, is highly functional in the correction of anatomical misalignments to thereby further enhance the cold related effect of the system. Accordingly, the invention, while in some respect resembling a traditional ice gel pack, is, through its unique density and geometry, able to conform to body anatomy and yet retain sufficient resilient to support body weight as is shown in FIGS. 2, 3, and 5 described above. The same will thereby be of value both as a self-help therapeutic aid and to massage therapists, chiropractors, physical therapists and orthopedists.

While there has been shown and described the preferred embodiment of the instant invention it is to be appreciated that the invention may be embodied otherwise than is herein specifically shown and described and that, within said embodiment, certain changes may be made in the form and arrangement of the parts without departing from the underlying ideas or principles of this invention as set forth in the claims appended herewith.

I claim:

1. A cryogenic therapeutic massage device, comprising:
a pressure-deformable gel, which may be cooled by freezing, said gel permanently encased in a flexible thermally conductive polymeric material to substantially define a tubular compress having a diameter in a range of about 3 to about 7 inches and a ratio of length-to-diameter in a range of about 2 to about 4.

2. The device as recited in claim 1, in which said gel encased within said flexible thermally conductive material, defines a density in a range of about 0.17 to about 0.25 ounces per cubic inch.

3. The device as recited in claim 1, in which said polymeric material comprises two layers, said layers comprising an inner layer having a low friction non-adhering communication with said gel and, adhered thereto, an outer layer having properties of biocompatibility with human skin.

4. The device as recited in claim 3, in which said gel encased within said flexible thermally conductive material, defines a density in a range of about 0.17 to about 0.25 ounces per cubic inch.

5. The device as recited in claim 2, in which a length of said device defines a range of about 12 to about 15 inches.

6. The device as recited in claim 3, in which a length of said device defines a range of about 12 to about 15 inches.

7. The device as recited in claim 4, in which a-length of said device defines a range of about 12 to about 15 inches.

8. The device as recited in claim 3, in which an aggregate thickness of both of said layers of material is in a range of about 2 to about 4 mils.

9. The device as recited in claim 4, further comprising:
a thick cloth sack surrounding said tubular compress to thereby preclude skin burn and to absorb moisture from skin against which the compress is applied.

10. The device as recited in claim 5, further comprising:
a thick cloth sack surrounding said tubular compress to thereby preclude skin burn and to absorb moisture from skin against which the compress is applied.

11. The device as recited in claim 9, in which said cloth sack comprises a Terry cloth fabric.
